(19) United States
(12) Patent Application Publication
(10) Pub. No.: US 2005/0272705 A1
(43) Pub. Date: Dec. 8, 2005

(54) Method of adminstering bisphosphonates

(76) Inventor: Victor Sloan, Scotch Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/531,676

(22) PCT Filed: Oct. 14, 2003
(86) PCT No.: PCT/EP03/11380

§ 371 Date: Apr. 15, 2005

(30) Foreign Application Priority Data
Oct. 15, 2002 (US) 60418555

(51) Int. Cl. A61K 31/675

(52) U.S. Cl. 514/94

(57) ABSTRACT

Bisphosphonates, in particular more potent N-bisphosphonates such as zoledronic acid and derivatives, can be used with satisfactory results for treatment of RA by intermittent administration, wherein the periods between bisphosphonate administrations are from about 2 months up to about 4 months, e.g. once every 3 months.

This invention relates to bisphosphonates, in particular to the pharmaceutical use of bisphosphonates in the treatment of rheumatoid arthritis (RA).

Our copending international patent application WO 01/97788 relates to the finding that bisphosphonates, in particular more potent nitrogen-containing bisphosphonates, can be used for prolonged inhibition of bone resorption in conditions of abnormally increased bone turnover by intermittent administration, wherein the period between administrations of bisphosphonate are longer than was previously considered appropriate to achieve satisfactory treatment, i.e. at intervals of at least about 6 months. We have now found that when bisphosphonates are used for the treatment of rheumatoid arthritis, optimal results are obtained if the bisphosphonate is dosed at frequency intervals less than 6 months.

Accordingly the present invention provides a method for the treatment of rheumatoid arthritis in a patient in need of such treatment which comprises intermittently administering an effective amount of a bisphosphonate to the patient, wherein the period between administrations of bisphosphonate is from at least about 2 months up to about 4 months.

The invention further provides use of a bisphosphonate in the preparation of a medicament for the treatment of rheumatoid arthritis in which the bisphosphonate is administered intermittently and in which the period between administrations of bisphosphonate is from at least about 2 months up to about 4 months.

The invention yet further provides a kit for the treatment of rheumatoid arthritis comprising one or more unit doses, each comprising an effective amount of a bisphosphonate, together with instructions for intermittent administration at intervals from at least about 2 months up to about 4 months.

Rheumatoid arthritis is a disease characterised by inflammation and swelling of skeletal joints, especially the small joints of the extremities, leading to erosion and destruction of cartilage and bone. The present invention may be used to inhibit, halt or even reverse the cartilage and bone erosion and destruction, and to decrease the pain, associated with rheumatoid arthritis.

Thus in the present description the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition

In accordance with the present invention the bisphosphonate dosing interval is at least about 2 months, e.g. once every 60 days, or less than about 4 months, e.g. once every 120 days, or at any interval therebetween. Most preferably the dosing interval is about 3 months, e.g. from about once every 80 days to about once every 100 days, especially about every 90 days or annual calendar quarter.

The bisphosphonates used in the present invention are typically those which inhibit bone resorption. Such compounds characteristically contain two phosphonate groups attached to a single carbon atom, forming a “P-C-P” structure, e.g. in a compound of formula I 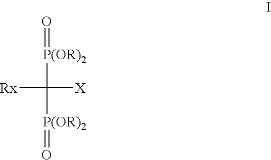
wherein
X is hydrogen, hydroxyl, amino, alkanoyl, or an amino group mono- or disubstituted by C₁-C₄ alkyl;
R is hydrogen or C₁-C₄ alkyl and
Rx is an optionally substituted hydrocarbyl group,
and pharmaceutically acceptable salts thereof or any hydrate thereof.

Thus, for example, suitable bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-di-methylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, (ibandronic acid), e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 1-(4-chlorophenylthio)methane-1,1-diphosphonic acid (tiludronic acid), e.g. tiludronate; 3-[N-(2-phenylthioethyl)-N-methylamino]-1-hydroxypropane-1,1-di-phosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-dipbosphonic acid tetraethyl ester, e.g. U-81581 (Upjohn); 1-hydroxy-2-(imidazo[1,2-a]pyridin-3-yl)etbane-1,1-diphosphonic acid, e.g. YM 529; and 1,1-dichloromethane-1,1-diphosphonic acid (clodronic acid), e.g. clodronate; YM175.

Preferred bisphosphonates for use in the present invention are N-bisphosphonates, i.e. compounds which in addition to the characteristic geminal bisphosphonates moiety (e.g. “P-C-P”) comprise a nitrogen-containing side chain, e.g. a compound of formula I′ 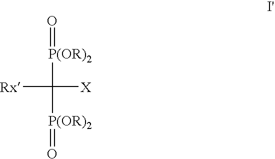
wherein
X is hydrogen, hydroxyl, amino, alkanoyl, or an amino group mono- or disubstituted by C₁-C₄ alkyl;
R is hydrogen or C₁-C₄ alkyl and
Rx′ is a side chain which contains an optionally substituted amino group, or a nitrogen containing heterocycle (including aromatic nitrogen-containing heterocycles),
and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-di-methylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenyl-aminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II 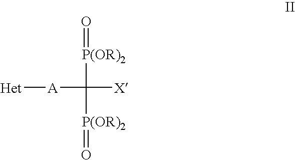
wherein
Het is an imidazole, oxazole, isoxazole, oxadiazole, thiazole, thiadiazole, pyridine, 1,2,3-triazole, 1,2,4-triazole or benzirnidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

In a further embodiment a particularly preferred bisphosphonate for use in the invention comprises a compound of Formula III 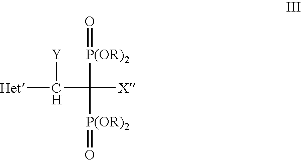
wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and
R is hydrogen or C₁-C₄ alkyl;
   as well as the pharmacologically acceptable salts and isomers thereof.

In a yet further embodiment a particularly preferred bisphosphonate for use in the invention comprises a compound of Formula IV 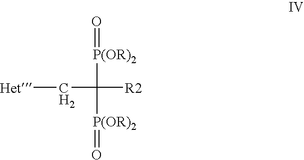
wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono-or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
   or a pharmacologically acceptable salt thereof.
   Examples of particularly preferred N-bisphophonates for use in the invention are:

2-(1-Methylimidazol-2-yl)-1-hydroxyethane-1,1-diphosphonic acid;

2-(1-Benzylimidazol-2-yl)-1-hydroxyethane-1,1-diphosphonic acid;

2-(1-Methylimidazol-4-yl)-1-hydroxyethane-1,1-diphosphonic acid;

1-Amino-2-(1-methylimidazol-4-yl)ethane-1,1-diphosphonic acid;

1-Amino-2-(1-benzylimidazol-4-yl)ethane-1,1-diphosphonic acid;

2-(1-Methylimidazol-2-yl)ethane-1,1-diphosphonic acid;

2-(1-Benzylimidazol-2-yl)ethane-1,1-diphosphonic acid;

2-(Imidazol-1-yl)-1-hydroxyethane-1,1-diphosphonic acid;

2-(Imidazol-1-yl)ethane-1,1-diphosphonic acid;

2-(4H-1,2,4-triazol-4-yl)-1-hydroxyethane-1,1-diphosphonic acid;

2-(Thiazol-2-yl)ethane-1,1-diphosphonic acid;

2-(Imidazol-2-yl)ethane-1,1-diphosphonic acid;

2-(2-Methylimidazol-4(5)-yl)ethane-1,1-diphosphonic acid;

2-(2-Phenylimidazol-4(5)-yl)ethane-1,1-diphosphonic acid;

2-(4,5-Dimethylimidazol-1-yl)-1-hydroxyethane-1,1-diphosphonic acid, and

2-(2-Methylimidazol-4(5)-yl)-1-hydroxyethane-1,1-diphosphonic acid,

and pharmacologically acceptable salts thereof.

The most preferred N-bisphosphonate for use in the invention is 2-(imidazol-1yl)-1-hydroxyethane-1,1-diphosphonic acid (zoledronic acid) or a pharmacologically acceptable salt thereof.

Pharmacologically acceptable salts are preferably salts with bases, conveniently metal salts derived from groups Ia, Ib, IIa and IIIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts is characterized by having one acidic hydrogen and one pharmaceutically acceptable cation, especially sodium, in each of the phosphonic acid groups.

The bisphosphonic acid derivatives specifically mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)-ethane-1,1-diphosphonic acid is prepared as described e.g. in U.S. Pat. No. 4,939,130.

The bisphosphonates (hereinafter referred to as the Agents of the Invention) may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The Agents of the Invention can also be used in the form of their hydrates or include other solvents used for their crystallisation.

The Agents of the Invention are preferably used in the form of pharmaceutical compositions that contain a therapeutically effective amount of active ingredient optionally together with or in admixture with inorganic or organic, solid or liquid, pharmaceutically acceptable carriers which are suitable for administration.

The Agents of the Invention may be administered alone or in combination with other bone active drugs, either in fixed combinations or separately both physically and in time, including hormones, such as a steroid hormone, e.g. an estrogen; a partial estrogen agonist, or estrogen-gestagen combination;, a calcitonin or an analogue or derivative thereof, e.g. salmon, eel or human calcitonin parathyroid hormone or analogues thereof, e.g. e.g. PTH (1-84), PTH (1-34), PTH (1-36), PTH (1-38), PTH (1-31)NH₂ or PTS 893; a SERM (Selective Estrogen Receptor Modulator) e.g. raloxifene, lasofoxifene, TSE-424, FC1271, Tibolone (Livial®); vitamin D or an analog. Such additional bone active drugs may be administered more frequently than the bisphosphonate.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially intravenous, subcutaneous, intramuscular, or transdermal) administration. Intravenous and oral, first and foremost intravenous administration is considered to be of particular importance. Preferably the bisphosphonate active ingredient is in the form of a parenteral, most preferably an intravenous form.

The particular mode of administration and the dosage may be selected by the attending physician taking into account the particulars of the patient, especially age, weight, life style, activity level, hormonal status (e.g. post-menopausal) and bone mineral density as appropriate.

The dosage of the Agents of the Invention may depend on various factors, such as effectiveness and duration of action of the active ingredient, e.g. including the relative potency of the bisphosphonate used, mode of administration, warm-blooded species, and/or sex, age, weight and individual condition of the warm-blooded animal.

Normally the dosage is such that a single dose of the bisphosphonate active ingredient from 0.005-20 mg/kg, especially 0.01-10 mg/kg, is administered to a warm-blooded animal weighing approximately 75 kg.

“mg/kg” means mg drug per kg body weight of the mammal—including man—to be treated.

The dose mentioned above is typically administered intermittently with a dosing interval which is at least about 2 months, e.g. once every 60 days, or less than about 4 months, e.g. once every 120 days, or at any interval therebetween. Most preferably the dosing interval is about 3 months, e.g. from about once every 80 days to about once every 100 days, especially about every 90 days or annual calendar quarter.

Formulations in single dose unit form contain preferably from about 1% to about 90%, and formulations not in single dose unit form contain preferably from about 0.1% to about 20%, of the active ingredient. Single dose unit forms such as ampoules of infusion solution or solid for preparation of infusion solution doses, capsules, tablets or dragees contain e.g. from about 0.5 mg to about 500 mg of the active ingredient. It will be appreciated that the actual unit dose used will depend upon the potency of the bisphosphonates, the dosing interval and route of administration amongst other things. Thus the size of the unit dose is typically lower for more potent bisphosphonates and greater the longer the dosing interval. For example, for more potent, N-bisphosphonates such as zoledronic acid a unit dose of from about 1 up to about 10 mg, preferably from about 3 mg up to about 7 mg, especially about 5 mg or about 4 mg may be used for parenteral, e.g. intravenous, administration. For example, also for more potent N-bisphosphonates which are dosed orally, e.g. alendronate, ibandronate or risedronate, an oral unit dose of from about 1 mg to about 100 mg, preferably from about 5 mg to about 70 mg, especially from about 10 mg to about 40 mg may be used.

Unit doses may be administered as a single or divided dose, i.e. a dose in which the unit dose is divided into two or more equal or unequal parts and in which the parts are administered to the patient at the same time, during overlapping time periods or at separate time points. When the unit dose is administered as a divided dose at separate time points, the interval between the separate administrations of the divided dose may be from hours, e.g. 1 hour, up to about 1 week (approximately 7 days). In accordance with the invention, the time interval between administration of the last part of the divided dose and administration of the first part of the next, following divided dose is at least about 2 months up to about 4 months, e.g. about 3 months.

In a particularly preferred embodiment a 5mg unit dose of zoledronic acid or salt thereof (dose based on free acid) is administered, e.g iv., once every 3 months.

In an alternative particularly preferred embodiment a 4 mg unit dose of zoledronic acid or salt thereof (dose based on free acid) is administered, e.g iv., once every 3 months.

Pharmaceutical preparations for enteral and parenteral administration are, for example, those in dosage unit forms, such as dragees, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Suitable carriers are especially fillers, such as sugars, for example lactose, saccharose, mannitol or sorbitol, cellulose preparations, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Colouring substances or pigments may be added to the tablets or dragee coatings, for example for the purpose of identification or to indicate different doses of active ingredient.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as, intramuscularly, intraperitoneally, intranasally, intradermally, subcutaneously or preferably intravenously. Such fluids are preferably isotonic aqueous solutions or suspensions which can be prepared before use, for example from lyophilised preparations which contain the active ingredient alone or together with a pharmaceutically acceptable carrier, or from solution concentrates. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient to the skin of the host at a controlled and predetermined rate over a prolonged period of time, and means to secure the device to the skin.

The invention includes kit products as hereinbefore defined, which characteristically comprise one or more unit doses, each comprising an effective amount of a bisphosphonate, together with instructions for intermittent administration. As is customary for pharmaceutical products these instructions for intermittent administration may be in the form of a package insert, or be present on the packaging orpackage labeling, or be available in any other form, including reference to an internet link or similar.

The following Examples illustrate the invention described hereinbefore.

In the following Examples the term “active ingredient” is to be understood as being any one of the bisphosphonic acid derivatives mentioned above as being useful according to the present invention.

### EXAMPLES

### Example 1

Capsules containing coated pellets of active ingredient, for example, disodium pamidronate pentahydrate, as active ingredient:

Core pellet:

| | | |
|---|---|---|
| active ingredient (ground) | 197.3 | mg |
| Microcrystalline cellulose | 52.7 | mg |
| (Avicel ® PH 105) | | |
| | 250.0 | mg |

+Inner coating:

| | | |
|---|---|---|
| Cellulose HP-M 603 | 10.0 | mg |
| Polyethylene glycol | 2.0 | mg |
| Talc | 8.0 | mg |
| | 270.0 | mg |

+Gastric juice-resistant outer coating:

| | | |
|---|---|---|
| Eudragit ® L 30 D (solid) | 90.0 | mg |
| Triethyl citrate | 21.0 | mg |
| Antifoam ® AF | 2.0 | mg |
| Water | | |
| Talc | 7.0 | mg |
| | 390.0 | mg |

A mixture of disodium pamidronate with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam® AF. The coated pellets are powdered with talc and filled into capsules (capsule size 0) by means of a commercial capsule filling machine, for example Höfliger and Karg.

### Example 2

Monolith adhesive transdermal system, containing as active ingredient, for example, 1-hydroxy-2-(imidazol-1 -yl)-ethane-1,1-diphosphonic acid:

Composition:

| | |
|---|---|
| polyisobutylene (PIB) 300 | 5.0 g |
| (Oppanol B1, BASF) | |
| PIB 35000 | 3.0 g |
| (Oppanol B1000, BASF) | |
| PIB 1200000 | 9.0 g |
| (Oppanol B100, BASF) | |
| hydrogenated hydrocarbon resin | 43.0 g |
| (Escorez 5320, Exxon) | |
| 1-dodecylazacycloheptan-2-one | 20.0 g |
| (Azone, Nelson Res., Irvine/CA) | |
| active ingredient | 20.0 g |
| Total | 100.0 g |

Preparation:

The above components are together dissolved in 150 g of special boiling point petroleum fraction 100-125 by rolling on a roller gear bed. The solution is applied to a polyester film (Hostaphan, Kalle) by means of a spreading device using a 300mm doctor blade, giving a coating of about 75 g/m². After drying (15 minutes at 60° C.), a silicone-treated polyester film (thickness 75 mm, Laufenberg) is applied as the peel-off film. The finished systems are punched out in sizes in the wanted form of from 5 to 30 cm² using a punching tool. The complete systems are sealed individually in sachets of aluminised paper.

### Example 3

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

Composition:

| | | |
|---|---|---|
| active ingredient (free diphosphonic acid) | 1.0 | mg |
| mannitol | 46.0 | mg |
| Trisodium citrate × 2H₂O | ca. 3.0 | mg |
| water | 1 | ml |
| water for injection | 1 | ml |

In 1 ml of water, the active ingredient is titrated with trisodium citrate×2 H₂O to pH 6.0. Then, the mannitol is added and the solution is lyophilized and the lyophilisate filled into a vial.

### Example 4

Ampoule containing active ingredient, for instance disodium pamidronate pentahydrate dissolved in water. The solution (concentration 3 mg/ml) is for i.v. infusion after dilution.

Composition:

| | | |
|---|---|---|
| active ingredient | 19.73 | mg |
| ( [symbol] 5.0 mg of anhydrous active ingredient) | | |
| mannitol | 250 | mg |
| water for injection | 5 | ml. |

### Example 5

### Treatment of Patients

Magnetic Resonance Imaging study (MRI)

A dose and dose regimen-finding 6 month study of zoledronic acid in patients with rheumatoid arthritis is carried out. 40 patients are randomised into 2 study arms. All patients are evaluated using imaging modalities at base line and at 3 monthly intervals. Patients that had recent exposure to bone active drugs, e.g bisphosphonates, estrogen, calcatonin, raloxifene, or a history of metabolic bone diseases are excluded. Zoledronic acid or placebo is administered as a bolus iv injection into a peripheral vein over about 5 minutes at every visit.

Efficacy is determined by measurement of percent change from base line in bone mineral density (BMD) measured by Magnetic Resonance Imaging as compared to MIX alone, at 3 and 6 months. Additionally, the number of new erosions on MRI by EULAR or OMERACT scoring system is measured.

The MRI study looks at 11 joints and 15 bones of the hand and wrist.

It is expected that the progression of erosions due to rheumatoid arthritis on treatment with MTX alone will be approximately 70%. In comparison the expected progression of erosions due to rheumatoid arthritis on treatment with zoledronic acid and MTX will be approximately 35%.

Study Arms
Methotrexate alone every three months
5 mg Zoledronic acid+Methotrexate every three months

X ray Study

A dose and dose regimen-finding 12 month trial of iv zoledronic acid in patients with rheumatoid arthritis is carried out. 200 patients are randomised into two study arms. Patients who had recent exposure to bone active drugs, e.g bisphosphonates, estrogen, calcitonin, raloxifene, or a history of metabolic bone diseases are excluded. All patients are evaluated with Methotrexate (MTX) as baseline and at 3-monthly intervals. Zoledronic acid and Methotrexate or Methotrexate is administered as a bolus iv injection into a peripheral vein over 5 minutes at every visit.

Efficacy is ascertained by measurement of percentage change in bone mineral density (BMD) measured by x-ray as compared to MIX and 6, and 12 months.

Additionally, the degree and duration of suppression of biochemical markers of bone turnover—Serum C-telopeptide (CTx), bone specific alkaline phosphate (BSAP)—is obtained every three months. Total sharp score (erosion and JSN), number of new patients with no new erosions, number of patients with sharp erosion score increase greater than 3, and ACR20 is also measured at three monthly intervals.

Study Arms
MTX only every three months
5 mg Zoledronic acid and MTX every three months

1. A method for the treatment of rheumatoid arthritis in a patient in need of such treatment which comprises intermittently administering an effective amount of a bisphosphonate to the patient, wherein the period between administrations of bisphosphonate is from at least about 2 months up to about 4 months.

2. (canceled)

3. A kit for the treatment of rheumatoid arthritis comprising oneor more unit doses, each comprising an effective amount of a bisphosphonate, according to a dose regimen for intermittent administration at intervals from at least about 2 months up to about 4 months to a patient in need of such treatment.

4. A method according to claim 1, in which the bisphosphonate dosing interval is from about once every 80 days to about once every 100 days.

5. A method according to claim 1, in which the bisphosphonate dosing interval is about once every 90 days or annual calendar quarter.

6. A method according to claim 1, in which the bisphosphonate is a compound of formula I′ 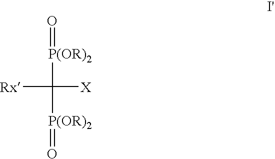
wherein
X is hydrogen, hydroxyl, amino, alkanoyl, or an amino group mono- or disubstituted by C₁-C₄ alkyl;
R is hydrogen or C₁-C₄ alkyl and
Rx′ is a side chain which contains an optionally substituted amino group, or a nitrogen containing heterocycle (including aromatic nitrogen-containing heterocycles),
and pharmaceutically acceptable salts thereof or any hydrate thereof.

7. A method according to claim 1, in which the bisphosphonate is a compound of formula IV 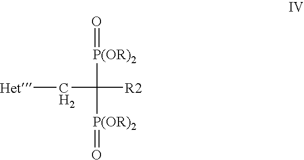
wherein
Het′″ is an imidazolyl, 2H-1,2,3-, 1 H-1,2,4- or 4H-1,2,4-triazolyl, tetrazolyl, oxazolyl, isoxazolyl, oxadiazolyl, thiazolyl or thiadiazolyl radical which is unsubstituted or C-mono-or di-substituted by lower alkyl, by lower alkoxy, bx phenyl which may in turn be mono- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
or a pharmacologically acceptable salt thereof.

8. A method, according to claim 7, in which the bisphosphonate is 1-hydroxy-2-(imidazol-1-yl)ethane-1,1-diphosphonic acid, or a pharmaceutically acceptable salt thereof, or any hydrate thereof.

9. A method, according to claim 8 in which 5 mg doses of zoledronic acid or salt thereof (dose based on free acid) are administered once every three months.
